(19) United States
(12) Patent Application Publication
(10) Pub. No.: US 2013/0053406 A1
(43) Pub. Date: Feb. 28, 2013

(54) THERAPEUTIC COMPOUNDS AND USES THEREOF

(71) Applicants: SE-HO KIM, Salt Lake City, UT (US); Herbert L. Ley, III, Salt Lake City, UT (US); Ashok Bajji, Salt Lake CIty, UT (US)

(72) Inventors: SE-HO KIM, Salt Lake City, UT (US); Herbert L. Ley, III, Salt Lake City, UT (US); Ashok Bajji, Salt Lake CIty, UT (US)

(73) Assignee: Myrexis, Inc., Salt Lake City, UT (US)

(21) Appl. No.: 12/861,721

(22) Filed: Aug. 23, 2010

Related U.S. Application Data
(60) Provisional application No. 61/236,021, filed on Aug. 21, 2009.

(51) Int. Cl. A61K 31/52; A61P 35/00; A61P 35/04; C07D 473/34

(52) U.S. Cl. 514/263.22; 544/276; 514/263.24

(57) ABSTRACT

The invention provides novel therapeutic compounds, pharmaceutical compositions comprising these compounds, and methods for using these compounds and compositions to treat diseases and disorders such as cancer.

### CROSS-REFERENCE TO RELATED APPLICATION

This application claims the benefit of U.S. Provisional Application No. 61/236,021, filed Aug. 21, 2009, which is incorporated by reference herein in its entirety.

### FIELD OF THE INVENTION

The invention relates to novel therapeutic compounds that inhibit Heat Shock Protein 90 (Hsp90). The invention also relates to pharmaceutical compositions comprising these compounds, and methods of treating diseases and disorders, such as cancers, that respond favorably to the inhibition of Hsp90.

### BACKGROUND OF THE INVENTION

Cancer is prevalent: Among United States citizens that live to be 70 years older and older, the probability of developing invasive cancer is 38% for females and 46% for males. According to the American Cancer Society, there will be about 1.4 million new cases of cancer in the United States alone in 2006. Although the five year survival rate for all cancers is now 65%, up from about 50% in the mid-nineteen seventies, cancer remains a leading killer today. Indeed, it is estimated that 565,000 people in the United States will die from cancer in 2006. (American Cancer Society, Surveillance Research, 2006). Although numerous treatments are available for various cancers, the fact remains that many cancers remain incurable, untreatable, and/or become resistant to standard therapeutic regimens. Thus, there is a clear need for new cancer treatments employing novel chemotherapeutic compounds.

Inhibitors of the molecular chaperone protein Hsp90 are being developed as one class of pharmacological weaponry in the anticancer chemotherapeutic arsenal. Consequently, there is a clear need for additional, novel, Hsp90 inhibitors for the treatment of diseases and disorders, such as cancer, that respond favorably to the inhibition of Hsp90.

### BRIEF SUMMARY OF THE INVENTION

In one aspect, the present invention comprises a compound according to Formula I:

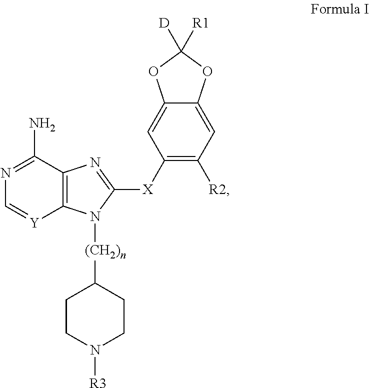

wherein

D is deuterium;

R1 is hydrogen or deuterium;

R2 is hydrogen, halo, hydroxyl, methoxy, trihalomethoxy, trihalomethyl, C₁₋₆ alkyl, —CH₂OH, CHF₂, CH₂F, cyano, nitro, amino, aminoalkyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, or N-thiocarbamyl;

R3 is hydrogen or —C(═O)R5, wherein R5 is selected from hydrogen, C₁₋₆ alkyl, aryl, and cycloalkyl, which are optionally substituted with one or more groups selected from halo, hydroxyl, thiol, alkylthio, arylthio, cyano, haloalkyl, alkoxy, amino, C-amido, N-amido, sulfonyl, sulfonamide, and heteroaryl;

X is CH₂, NH, NCH₃, NCH₂CH₃, NCH(CH₃)₂, O, or S;

Y is N or CH; and

n is 0 to 4;

and pharmaceutically acceptable salts thereof.

In another aspect, the present invention comprises a compound according to Formula II:

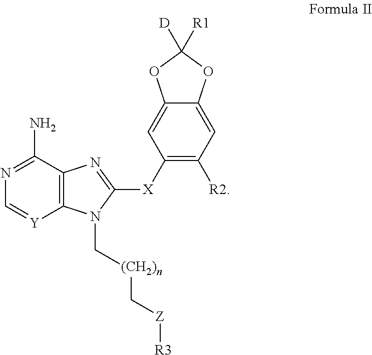

wherein

D is deuterium;

R1 is hydrogen or deuterium;

R2 is hydrogen, halo, hydroxyl, methoxy, trihalomethoxy, trihalomethyl, C₁₋₆ alkyl, —CH₂OH, CHF₂, CH₂F, cyano, nitro, amino, aminoalkyl, C-amido N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, or N-thiocarbamyl;

R3 is hydrogen, alkyl, alkenyl, alkynyl, amino, cyano, carbocycle, or heterocycle;

X is CH₂, NH, NCH₃, NCH₂CH₃, NCH(CH₃)₂, O, or S;

Y is N or CH;

Z is optionally present, and when present is O, S, CH₂, CHR4, NH, or NR4, wherein R4 is independently selected from H, alkyl, carbocycle, heterocycle, amino, aminoalkyl, carbonyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, and N-thiocarbamyl; and n is 0 to 3;

and pharmaceutically acceptable salts thereof.

The compounds of the present invention include the compounds of the Formula I and Formula II as illustrated herein, as well as their geometric isomers, enantiomers, diastereomers, or racemates thereof. The compounds of the present invention also include pharmaceutically acceptable salts, esters, prodrugs and solvates of all such compounds.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

### 1. Definitions

The term “bioisostere,” as used herein, generally refers to compounds or moieties that have chemical and physical properties producing broadly similar biological properties. For example, —COOH bioisosteres include, but are not limited to, a carboxylic acid ester, amide, tetrazole, oxadiazole, isoxazole, hydroxythiadiazole, thiazolidinedione, oxazolidinedione, sulfonamide, sulfonylcarboxamide, phosphonic acid, phosphonamide, phosphinic acid, sulfonic acid, acyl sulfonamide, mercaptoazole, and cyanamide.

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). An alkyl group may be in unsubstituted form or substituted form with one or more substituents (generally one to three substitutents except in the case of halogen substituents, e.g., perchloro). For example, a C₁₋₆ alkyl group (“lower alkyl”) refers to a straight or branched aliphatic group containing 1 to 6 carbon atoms (e.g., but not limited to, methyl, ethyl, propyl, isopropyl, sec-butyl, tent-butyl, isobutyl, n-butyl, 3-pentyl, and hexyl), which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. An alkenyl group may be in unsubstituted form or substituted form with one or more substituents (generally one to three substitutents except in the case of halogen substituents, e.g., perchloro or perfluoroalkyls). For example, a C₁₋₆ alkenyl group refers to a straight or branched chain radical containing 1 to 6 carbon atoms and having at least one double bond between two of the carbon atoms in the chain (e.g., but not limited to, ethenyl, 1-propenyl, 2-propenyl, 2-methyl-l-propenyl, 1-butenyl and 2-butenyl, which may be optionally substituted).

The term “alkynyl” as used herein by itself or as part of another group means a straight or branched chain radical of 2-10 carbon atoms, unless the chain length is limited thereto, wherein there is at least one triple bond between two of the carbon atoms in the chain. An alkynyl group may be in unsubstituted form or substituted form with one or more substituents (generally one to three substitutents except in the case of halogen substituents, e.g., perchloro or perfluoroalkyls). For example, a C₁₋₆ alkynyl group refers to a straight or branched chain radical containing 1 to 6 carbon atoms and having at least one triple bond between two of the carbon atoms in the chain (e.g., but not limited to, ethynyl, 1-propynyl, 1-methyl-2-propynyl, 2-propynyl, 1-butynyl and 2-butynyl, which may be optionally substituted).

The term “carbocycle” as used herein by itself or as part of another group means cycloalkyl and non-aromatic partially saturated carbocyclic groups such as cycloalkenyl and cycloalkynyl. A carbocycle may be in unsubstituted form or substituted form with one or more substituents so long as the resulting compound is sufficiently stable and suitable for the treatment method of the present invention.

The term “cycloalkyl” as used herein by itself or as part of another group refers to a fully saturated 3- to 8-membered cyclic hydrocarbon ring (i.e., a cyclic form of an unsubstituted alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, polycyclic or a spiral ring. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl may be in unsubstituted form or substituted form with one or more substituents so long as the resulting compound is sufficiently stable and suitable for the treatment method of the present invention. Non-limiting examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring (i.e., a cyclic form of an unsubstituted alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is recited as a substituent on a chemical entity, it is intended that the cycloalkenyl moiety is attached to the entity through a carbon atom within the fully saturated cyclic hydrocarbon ring of the cycloalkenyl. In contrast, a substituent on a cycloalkenyl can be attached to any carbon atom of the cycloalkyl. A cycloalkenyl group may be unsubstituted or substituted with one or more substitutents. Non-limiting examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3-7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of carbon atoms with such other rings) (“polycyclic heterocycle”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocycle” contains one or more double or triple bonds within the non-aromatic heteroatom-containing cyclic ring regardless of the other ring it is fused to. A heterocycle may be in unsubstituted form or substituted form with one or more substituents so long as the resulting compound is sufficiently stable and suitable for the treatment method of the present invention.

Some non-limiting examples of saturated or partially saturated heterocyclic groups include furanyl, tetrahydrofuranyl, thiophenyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocyclic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be in unsubstituted form or substituted form with one or more substituents so long as the resulting compound is sufficiently stable and suitable for the treatment method of the present invention.

The term “heteroaryl” as employed herein refers to a stable aromatic ring having up to 7 atoms with 1, 2, 3 or 4 heteroatoms which are oxygen, nitrogen or sulfur or a combination thereof (“monocyclic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be in unsubstituted form or substituted form with one or more substituents so long as the resulting compound is sufficiently stable and suitable for the treatment method of the present invention.

Non-limiting examples of heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-amino-isocoumarin, pyrido[1,2-a]pyrimidin-4-one, pyrazolo[1,5-a]pyrimidinyl, including without limitation pyrazolo[1,5-a ]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to chloro, fluoro, bromo, and iodo.

As used herein, the term “hydro” refers to a hydrogen atom (—H group).

As used herein, the term “hydroxyl” refers to an -OH group.

As used herein, unless otherwise specified, the term “alkoxy” refers to a —O—C₁₋₁₂ alkyl.

As used herein, the term “cycloalkyloxy” refers to an —O-cycloalkyl group.

As used herein, the term “aryloxy” refers to an —O -aryl group.

As used herein, the term “heteroaryloxy” refers to both an —O-heteroaryl group.

Non-limiting examples of acyloxy groups include any C₁₋₆ acyl (alkanoyl) attached to an oxy (—O—) group, e.g., formyloxy, acetoxy, propionoyloxy, butanoyloxy, pentanoyloxy and hexanoyloxy. An acyloxy group may be unsubstituted or substituted form with one or more substituents so long as the resulting compound is sufficiently stable and suitable for the treatment method of the present invention.

As used herein, the term “mercapto” group refers to an —SH group.

As used herein, the term “alkylthio” group refers to an —S-alkyl group. Non-limiting examples of alkythio include SCH₃.

As used herein, the term “arylthio” group refers to both an —S-aryl group.

The term “arylalkyl” is used herein to mean an above-defined alkyl group substituted by an aryl group defined above. Non-limiting examples of arylalkyl groups include benzyl, phenethyl and naphthylmethyl, etc. An arylalkyl group may be unsubstituted or substituted with one or more substituents so long as the resulting compound is sufficiently stable and suitable for the treatment method of the present invention.

The term “heteroarylalkyl” is used herein to mean an alkyl group defined above substituted by any heteroaryl groups. A heteroarylalkyl may be unsubstituted or substituted with one or more substituents so long as the resulting compound is sufficiently stable and suitable for the treatment method of the present invention.

The term “arylalkenyl” is used herein to mean an alkenyl group defined above substituted by any aryl groups defined above.

The term “heteroarylalkenyl” is used herein to mean any of the above-defined alkenyl groups substituted by any of the above-defined heteroaryl groups.

The term “arylalkynyl” is used herein to mean any of the above-defined alkynyl groups substituted by any of the above-defined aryl groups.

The term “heteroarylalkynyl” is used herein to mean any of the above-defined alkynyl groups substituted by any of the above-defined heteroaryl groups.

The term “aryloxy” is used herein to mean aryl-O— wherein aryl is as defined above. Non-limiting examples of aryloxy groups include phenoxy and 4-methylphenoxy.

The term “heteroaryloxy” is used herein to mean heteroaryl-O— wherein heteroaryl is as defined above.

The term “arylalkoxy” is used herein to mean an alkoxy group substituted by an aryl group as defined above. Non-limiting examples of arylalkoxy groups include benzyloxy and phenethyloxy.

“Heteroarylalkoxy” is used herein to mean any of the above-defined alkoxy groups substituted by any of the above-defined heteroaryl groups.

“Haloalkyl” means an alkyl group substituted by one or more (1, 2, 3, 4, 5 or 6) fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Non-limiting examples of acylamino (acylamido) groups include any C₁₋₆ acyl (alkanoyl) attached to an amino nitrogen which is in turn attached to the main structure, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein.

As used herein, the term “O-carboxy” group refers to a R″C(═O)O-group, with R″ as defined herein.

As used herein, the term “C-carboxy” group refers to a —C(═O)OR″ groups with R″ as defined herein.

As used herein, the term “ester” is a C-carboxy group, as defined herein, wherein R″ defined above except that it is not hydro (e.g., methyl, ethyl, lower alkyl).

As used herein, the term “C-carboxy salt” refers to a —C(═O)O⁻M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “trihalomethanesulfonyl” refers to a X₃ CS(═O)₂-group with X is a halo as defined above.

As used herein, the term “cyano” refers to a —C≡N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂R″ group with R″ as defined herein. Non-limiting examples of sulfonyl groups include —SO₂CH₃.

As used herein, the term “sulfonamide” refers to a —S(═O)₂N(R¹⁷)(R¹⁸), with R¹⁷ and R¹⁸ as defined herein. Non-limiting examples of sulfonamide groups include —SO₂NH₂, SO₂NHCH₃, SO₂N(CH₃)₂).

As used herein, the term “trihalomethanesulfonamido” refers to a X₃CS(═O)₂ NR¹⁷-group with X is halo as defined above and R¹⁷ as defined herein.

As used herein, the term “O-carbamyl” refers to a —OC(═O)N(R¹⁷)(R¹⁸) group with R¹⁷ and R¹⁸ as defined herein.

As used herein, the term “N-carbamyl” refers to a R¹⁸OC(═O)NR¹⁷— group, with R¹⁷ and R¹⁸ as defined herein.

As used herein, the term “O-thiocarbamyl” refers to a —OC(═S)N(R¹⁷)(R¹⁸) group with R¹⁷ and R¹⁸ as defined herein.

As used herein, the term “N-thiocarbamyl” refers to a R¹⁷OC(═S)NR¹⁸— group, with R¹⁷ and R¹⁸ as defined herein.

As used herein, the term “amino” refers to an —N(R¹⁷)(R¹⁸) group, with R¹⁷ and R¹⁸ as defined herein. Non-limiting examples of amino groups include NH₂, NHCH₃, N(CH₃)₂, NHCH₂CH₃, N(CH₂CH₃)₂, NHCH(CH₃)CH₃, N(CH(CH₃)CH₃)₂, NHCH(CH₂CH₃)₂, N(CH(CH₂CH₃)₂)₂.

As used herein, the term “aminoalkyl” refers to a moiety wherein an amino group as defined herein attached through the nitrogen atom to an alkyl group as defined above.

As used herein, the term “C-amido” refers to a —C(═O)N(R¹⁷)(R¹⁸) group with R¹⁷ and R¹⁸ as defined herein. An “N-amido” refers to a R¹⁷C(═O)NR¹⁸— group or a R″C(═O)NR¹⁸— group with R″, R¹⁷, and R¹⁸ as defined herein. Non-limiting examples of N-amido groups include NHC(═O)CH(OH)CH₃.

As used herein, the term “C-amidoalkyl” refers to a —C₁₋₆ alkyl-CO₂N(R¹⁷)(R¹⁸) group with R¹⁷ and R¹⁸ as defined herein.

As used herein, the term “nitro” refers to a —NO₂ group.

As used herein, the term “quaternary ammonium” refers to a —⁺N(R¹⁷)(R¹⁸)(R¹⁹) group wherein R¹⁷, R¹⁸, and R¹⁹ are as defined herein.

R¹⁷, R¹⁸, and R¹⁹ are independently selected from the group consisting of hydro and unsubstituted lower alkyl.

As used herein, the term “methylenedioxy” refers to a —OCH₂O— group wherein the oxygen atoms are bonded to adjacent ring carbon atoms.

As used herein, the term “ethylenedioxy” refers to a —OCH₂CH₂O— group wherein the oxygen atoms are bonded to adjacent ring carbon atoms.

### 2. Therapeutic Compounds

In one aspect, the present invention comprises a compound according to Formula I:

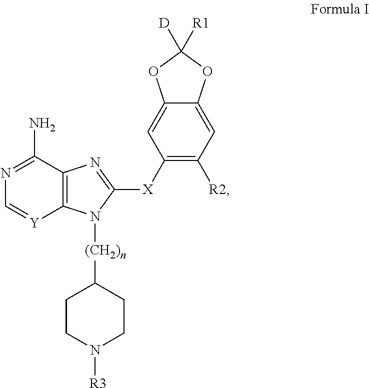

wherein

D is deuterium;

R1 is hydrogen or deuterium;

R2 is hydrogen, halo, hydroxyl, methoxy, trihalomethoxy, trihalomethyl, C₁₋₆ alkyl, —CH₂OH, CHF₂, CH₂F, cyano, nitro, amino, aminoalkyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, or N-thiocarbamyl;

R3 is hydrogen or —C(═O)R5, wherein R5 is selected from hydrogen, C₁₋₆ alkyl, aryl, and cycloalkyl, which are optionally substituted with one or more groups selected from halo, hydroxyl, thiol, alkylthio, arylthio, cyano, haloalkyl, alkoxy, amino, C-amido, N-amido, sulfonyl, sulfonamide, and heteroaryl;

X is CH₂, NH, NCH₃, NCH₂CH₃, NCH(CH₃)₂, O, or S;

Y is N or CH; and

n is 0 to 4

and pharmaceutically acceptable salts thereof.

It should be understood that when n is 0, the imidazole ring is linked directly (i.e., with no intervening methylene groups) to the piperidine ring of the compounds of Formula I.

In some embodiments of this aspect of the invention n is 2.

In some embodiments of this aspect of the invention n is 3.

In some embodiments of this aspect of the invention Y is N.

In some embodiments of this aspect of the invention, R2 is F, Cl, Br, or I.

In some embodiments of this aspect of the invention, R2 is N(CH₃)₂.

In some embodiments of this aspect of the invention, the R3 substituent is:

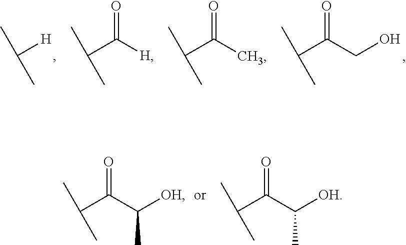

In another aspect, the present invention comprises a compound according to Formula II:

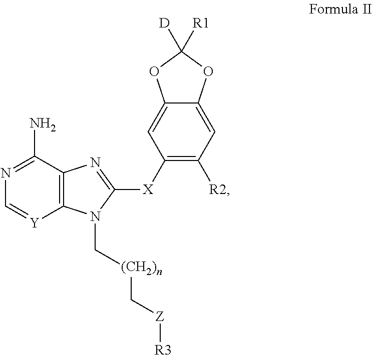

wherein

D is deuterium;

R1 is hydrogen or deuterium;

R2 is hydrogen, halo, hydroxyl, methoxy, trihalomethoxy, trihalomethyl, C₁-₆ alkyl, —CH₂OH, CHF₂, CH₂F, cyano, nitro, amino, aminoalkyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, or N-thiocarbamyl;

R3 is hydrogen, alkyl, alkenyl, alkynyl, amino, cyano, carbocycle, or heterocycle;

X is CH₂, NH, NCH₃, NCH₂CH₃, NCH(CH₃)₂, O, or S;

Y is N or CH;

Z is optionally present, and when present is O, S, CH₂, CHR4, NH, or NR4, wherein R4 is independently selected from H, alkyl, carbocycle, heterocycle, amino, aminoalkyl, carbonyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, and N-thiocarbamyl; and

n is 0 to 3

and pharmaceutically acceptable salts thereof.

In some embodiments of this aspect of the invention n is 0.

In some embodiments of this aspect of the invention n is 1.

In some embodiments of this aspect of the invention Y is N.

In some embodiments of this aspect of the invention Y is CH.

In some embodiments of this aspect of the invention, R2 is F, Cl, Br, or I.

In some embodiments of this aspect of the invention, R2 is N(CH₃)₂.

In some embodiments of this aspect of the invention, Z is NH.

In some embodiments of this aspect of the invention, Z is CHR4.

In some embodiments of this aspect of the invention, the R3 substituent is isopropyl, tert-butyl, or 2,2-dimethyl propyl.

In some embodiments of this aspect of the invention, the R3 substituent is NH₂.

In some embodiments of this aspect of the invention, the R3 substituent is:

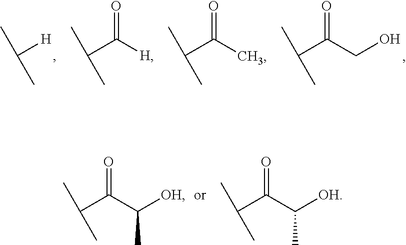

In some embodiments of this aspect of the invention, Z is CHR4 and R4 is cyclopropyl, cyclobutyl, cyclopentyl, cyclohexyl, or heterocycle.

The compounds of the present invention include the compounds of the Formulae I and II as illustrated herein, as well as their geometric isomers, enantiomers, diastereomers, or racemates thereof. The compounds of the present invention may contain one or more chiral centers, in each of which, the chiral center may be (R), or (S) configuration, or a mixture of both. The compounds of the present invention also include pharmaceutically acceptable salts, esters, prodrugs and solvates of all such compounds.

In preferred embodiments, compounds of Formulae I and II, having an IC₅₀ of less than 2,500 nM, 500 nM, 300 nM, 200 nM, preferably less than 100 nM, and most preferably less than 50 nM, as determined by the Hsp90 binding assay, which is described in the “Biological and Pharmacological Assays and Examples” section below, are used as the therapeutic compounds of the invention.

In the compounds of the invention, reference to any bound hydrogen atom can also encompass a deuterium atom bound at the same position. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibits a significantly longer half-life in the bodies of individuals administered such compounds. This is particularly so when the catabolism of the hydrogenated compound is mediated by cytochrome P450 systems. Kushner et al., Can. J. Physiol. Pharmacol. (1999) 77:79-88, the contents of which are incorporated herein in their entirety.

As used herein, the phrase “treating . . . with . . . a compound” means either administering the compound to cells or an animal, or administering to cells or an animal another agent to cause the presence or formation of the compound inside the cells or the animal. Preferably, the methods of the present invention comprise administering to cells in vitro or to a warm-blood animal, particularly mammal, and more particularly a human, a pharmaceutical composition comprising an effective amount of a compound according to the present invention.

A pharmaceutically acceptable salt of the compound of the present invention is exemplified by a salt with an inorganic acid and/or a salt with an organic acid that are known in the art. In addition, pharmaceutically acceptable salts include acid salts of inorganic bases, such as salts containing alkaline cations, alkaline earth cations, as well as acid salts of organic bases. Hydrates, solvates, and the like are also encompassed by the compounds of the present invention. In addition, N-oxide compounds are also encompassed in the compound of the present invention.

Additionally, as implied above, the compounds of the present invention can contain asymmetric carbon atoms and can therefore exist in racemic and optically active forms. Thus, optical isomers or enantiomers, racemates, and diastereomers of the depicted compounds are also encompassed. The methods of present invention include the use of all such isomers and mixtures thereof. The present invention encompasses any isolated racemic or optically active form of compounds described above, or any mixture thereof, which possesses therapeutic activity, particularly anti-cancer activity.

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

### 3. Pharmaceutical Compositions

In another aspect, the present invention further provides a medicament or a pharmaceutical composition having a therapeutically or prophylactically effective amount of a therapeutic compound according to the present invention.

Typically, therapeutic compounds according to the present invention can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for each administration can be, e.g., from about 1 μg to about 2000 mg, preferably from about 5 μg to about 1000 mg. In the case of combination therapy, a therapeutically effective amount of one or more other anticancer compounds can be administered in a separate pharmaceutical composition, or alternatively included in the pharmaceutical composition according to the present invention which contains a compound according to the present invention. The pharmacology and toxicology of many of such other anticancer compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In the pharmaceutical compositions, the active agents can be in any pharmaceutically acceptable salt form. As used herein, the term “pharmaceutically acceptable salts” refers to the relatively non-toxic, organic or inorganic salts of the active compounds, including inorganic or organic acid addition salts of the compound.

For oral delivery, the active compounds can be incorporated into a formulation that includes pharmaceutically acceptable carriers such as binders, lubricants, disintegrating agents, and sweetening or flavoring agents, all known in the art. The formulation can be orally delivered in the form of enclosed gelatin capsules or compressed tablets. Capsules and tablets can be prepared using any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included.

The active compounds can also be administered parenterally in the form of solution or suspension, or in lyophilized form capable of conversion into a solution or suspension form before use. In such formulations, diluents or pharmaceutically acceptable carriers such as sterile water and buffered physiological saline can be used. Other conventional solvents, buffers, stabilizers, anti-bacteria agents, surfactants, and antioxidants can all be included. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. See, e.g., Phillips et al., J. Pharmaceut. Sci., 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

In some embodiments of the pharmaceutical formulations of the present invention, pharmaceutically acceptable solubilizing agent are employed along with other excipients and carriers to create the pharmaceutical composition comprising one or more compounds of Formulae I and II. In such instances the solubilizing agent may be selected from cyclodextrins, liposomes, thermodynamically stable colloidal dispersions (e.g. micelles or microemulsions) containing surface-active agents, emulsions, or mixtures thereof. Typically, cyclodextrin solubilizing agents are water-soluble β-cyclodextrin derivatives, including sulfobutyl ether β-cyclodextrin, 2-hydroxypropyl β-cyclodextrin, or any other suitably chemically modified β-cyclodextrin, or any mixture thereof. Liposomes can reduce the toxicity of the active compounds, and increase their stability. Liposomes can also be used as carriers for the active compounds of the present invention. Liposomes are micelles made of various lipids such as cholesterol, phospholipids, fatty acids, and derivatives thereof. Various modified lipids can also be used. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976). When liposomes are utilized as a solubilizing agent, typically they will be formulated as an acidic liposomal suspension having a pH of up to about 6.0. Suitable thermodynamically stable colloidal dispersions may be formulated to have a pH of up to about 6.0, and may comprise a mixture of a surfactant lipid such as Cremophor EL, Vitamin E TPGS, various Pluronics (e.g., polyethylene oxide/polypropylene oxide polymers, Tween 80), and the like. The dispersions may also contain a water miscible co-solvent selected from ethanol, polyethylene glycol (PEG), propylene glycol (PG), glycerol, or any mixture thereof. Dextrose or other suitable excipients may be added to adjust tonicity.

The active compounds can also be administered in combination with another active agent that synergistically treats or prevents the same symptoms or is effective for another disease or symptom in the patient treated, so long as the other active agent does not interfere with, or adversely affect, the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

### 4. Therapeutic Methods

The present invention provides therapeutic methods comprising administering to an animal (e.g., a patient, in need of such treatment) a therapeutically effective amount of one or more compounds of Formula I and II, as defined above, and/or a pharmaceutically acceptable salt thereof. The therapeutic methods are particularly useful in the treatment of Hsp90 inhibitor-sensitive cancers, which comprise a group of diseases characterized by the uncontrolled growth and spread of abnormal cells. Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In another aspect, the invention provides a method for treating an individual having an Hsp90 inhibitor-sensitive disease or disorder chosen from inflammatory diseases, infections, autoimmune disorders, stroke, ischemia, cardiac disorders, neurological disorders, proliferative disorders, neoplasms, malignant diseases, and metabolic diseases.

In yet another aspect, the invention provides a method for treating an individual having an Hsp90 inhibitor-sensitive fibrogenetic disorder, such as, for example, scleroderma, polymyositis, systemic lupus, rheumatoid arthritis, liver cirrhosis, keloid formation, interstitial nephritis and pulmonary fibrosis.

### EXAMPLES

### Chemical Synthesis and Purification of Example Compounds

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Otherwise their preparation is facile and known to one of ordinary skill in the art, or it is referenced or described herein. Yields are not optimized. The chemical names were mostly generated using the ACD labs software (Version 8.08) available from Advanced Chemistry Development, Inc. (Toronto, Ontario, Canada), or the “Autonom 2000” plug-in for the Isis™/Draw 2.5SP1 chemical drawing program, available from MDL Information Systems, a division of Symyx technologies, Inc. (Santa Clara, Calif.). However, portions of the names describing the location of deuterium atoms have been chosen to conform to art-accepted practices.

Analytical TLC plates (Silica Gel 60 F254, EM Science, Merck 5715-7, EM Science, Gibbstown, N.J.) were used to follow the course of reactions, and the MPLC system used for purifications (Isco Foxy Jr fraction collector, UA-6 detector) was from Teledyne Isco, Inc. (Lincoln, Nebr.), using Isco silica gel flash columns. ¹H NMR spectra were recorded on a Varian Mercury 400 MHz instrument (Varian Inc., Polo Alto, Calif.) and chemical shifts are expressed in parts per million (ppm, δ) relative to TMS as the internal standard. Mass spectra were obtained on an Agilent LC/MS TOF, injection volume 2 uL, XTerra MS-C₁₈ 3.5 μm 2.1×50 mm column (Agilent Technologies, Santa Clara, Calif.) ESI source. Analytical HPLC was performed on an HP1100 (Agilent Technologies, Santa Clara, Calif.) injection volume 5 μl, Waters (Waters Corporation, Milford, Mass.) XBridge C,₈ 5 μm 4.6×150 mm column. Preparative HPLC purifications were performed using either Agilent HP-1100 preparative LC. The sample preparations and conditions were described below.

Method A: Agilent HP-1100 preparative LC:

Samples were dissolved in dimethylsulfoxide and resolved with a Waters XTera prep MS C₁₈ column (Waters Corporation, Milford, Mass.) 19×250 mm, 10 μparticle was used. The column was eluted with a mixture of acetonitrile and water (both containing 0.01% v/v trifluoroacetic acid) in a flow rate of 30 mL/min and a gradient of 25% 100% methanol over a period of 20 min.

### Abbreviations and Acronyms

When the following abbreviations are used herein, they have the following meaning:

| | |
|---|---|
| Ac₂O | acetic anhydride |
| anhy | Anhydrous |
| n-BuOH | n-butanol |
| t-BuOH | t-butanol |
| CD₃OD | methanol-d₄ |
| Celite ® | diatomaceous earth filter agent, ® Celite Corp. |
| CH₂Cl₂ | methylene chloride |
| DCM | dichloromethane |
| CI-MS | chemical ionization mass spectroscopy |
| conc | concentrated |
| dec | decomposition |
| bs | broad singlet |
| br | broad |
| DME | dimethoxyethane |
| DMF | N,N-dimethylformamide |
| DMSO | dimethylsulfoxide |
| DMSO-d₆ | dimethylsulfoxide-d₆ |
| ELSD | evaporative light scattering device |
| EtOAc | ethyl acetate |
| EtOH | ethanol (100%) |
| Et₂O | diethyl ether |
| Et₃N | triethylamine |
| HPLC ESI-MS | high performance liquid chromatography-electrospray |
| | mass spectroscopy |
| MPLC | medium pressure liquid chromatography |
| NMR | nuclear magnetic resonance spectroscopy |
| TOF-MS | time-of-flight-mass spectroscopy |
| NMM | 4-methylmorpholine |
| Ph₃P | triphenylphosphine |
| Pd(dppf)Cl₂ | [1,1′- |
| | bis(diphenylphosphino)ferrocene]dichloropalladium(II) |
| Pd(PPh₃)₄ | tetrakis(triphenylphosphine)palladium(0) |
| Pd(OAc)₂ | palladium(II) acetate |
| P(O)Cl₃ | phosphorous oxychloride |
| R_{f} | TLC retention factor |
| RT | retention time (HPLC) |
| rt | room temperature |
| THF | tetrahydrofuran |
| TFA | trifluoroacetic acid |
| TLC | thin layer chromatography |
| LC-MS (ESI) | liquid chromatography-mass spectroscopy (electrospray |
| | ionization) |
| DIEA | diisopropylethylamine |
| TFAA | trifluoroacetic anhydride |
| MsCl | methanesulfonylchloride |
| AcOH | acetic acid |
| HCl | hydrochloric acid |
| H₂SO₄ | sulfuric acid |
| HNO₃ | nitric acid |
| HBr | hydrobromic acid |
| CDCl₃ | chloroform-d₃ |
| CHCl₃ | chloroform |
| H₂O | water |
| NaOAc | sodium acetate |
| KOH | potassium hydroxide |
| NaOH | sodium hydroxide |
| NaCl | sodium chloride |
| NaHCO₃ | sodium bicarbonate |
| Na₂CO₃ | sodium carbonate |
| K₂CO₃ | potassium carbonate |
| Na₂SO₄ | sodium sulfate |
| MgSO₄ | magnesium sulfate |
| MeOH | methanol |
| SiO₂ | silica gel |
| K₃PO₄ | potassium phosphate |
| NH₄Cl | ammonium chloride |
| AIBN | 2,2′-axo bisisobutyronitrile |
| Barton's base | 2-t-butyl-1,1,3,3-tetramethylguanidine |
| DMAP | N,N-Dimethyl aminopyridine |
| LG | leaving group |
| MsCl | methanesulfonyl chloride |
| TsCl | p-toluenesulfonyl chloride |
| PG | protecting group |
| Xantphos | 4,5-bis(diphenylphosphino)-9,9-dimethyl xanthane |

### Methods of Synthesis

General methods, according to some embodiments, for the preparation of compounds of the present invention are provided in Schemes I and II below.

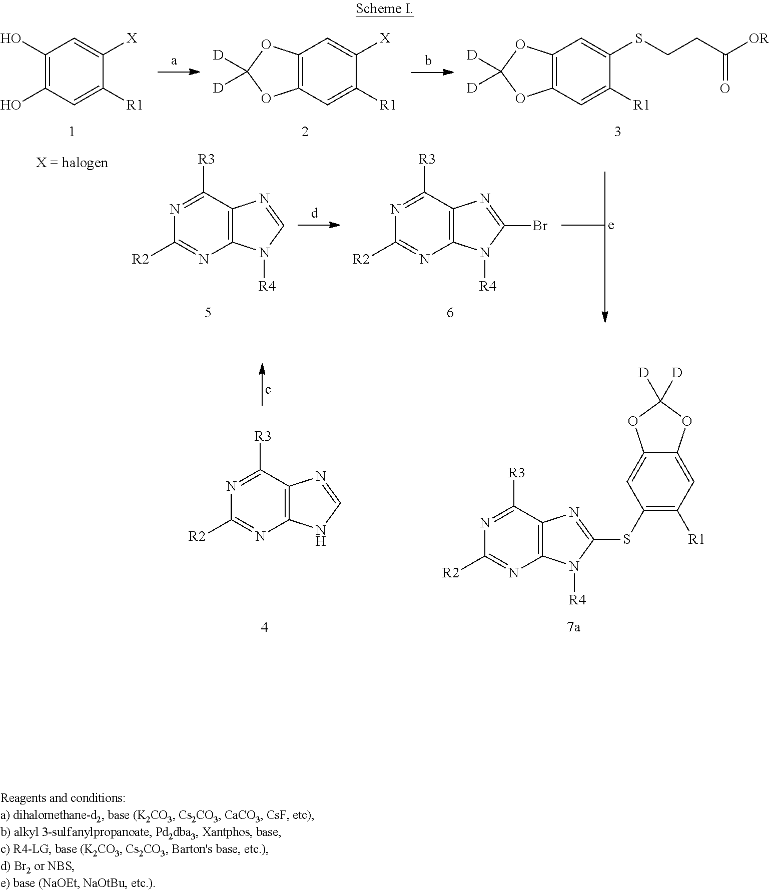

A general synthesis of compound 7a, according to some embodiments of the present invention, is outlined in Scheme 1. This approach involves a two-step synthesis of deuterated compound 3, which consist of the formation of d₂-benzo[d][1,3]dioxole 2 by the reaction of catechol 1 with deuterated dihalomethane in the presence of base (e.g., Cs₂CO₃, K₂CO₃, CsF). The subsequent palladium catalyzed coupling reaction of 2 with alkyl 3-sufanylpropanoate, forms the thiol surrogate (J. Org. Chem. 2006, 71, 2203, the contents of which are incorporated herein in their entirety). The R1 substituent in compound 3 represents various groups such as, for example, hydrogen, halo, hydroxyl, methoxy, trihalomethoxy, trihalomethyl, C1-C6 alkyl, —CH₂OH, CHF₂, CH₂F, cyano, nitro, amino, aminoalkyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, and N-thiocarbamyl. Bromoadenine 6 can be prepared by the previously described method (WO 2009/06503), the contents of which are incorporated herein in their entirety, starting from 4 in two steps; alkylation and followed by bromination. Finally treatment of 3 with an appropriate base can generate the corresponding thiolate anion which then could react with bromoadenine 6 to afford 7a. Depending on the functional group present in R4, compound 7a can be further elaborated to generate a wide variety of target molecules.

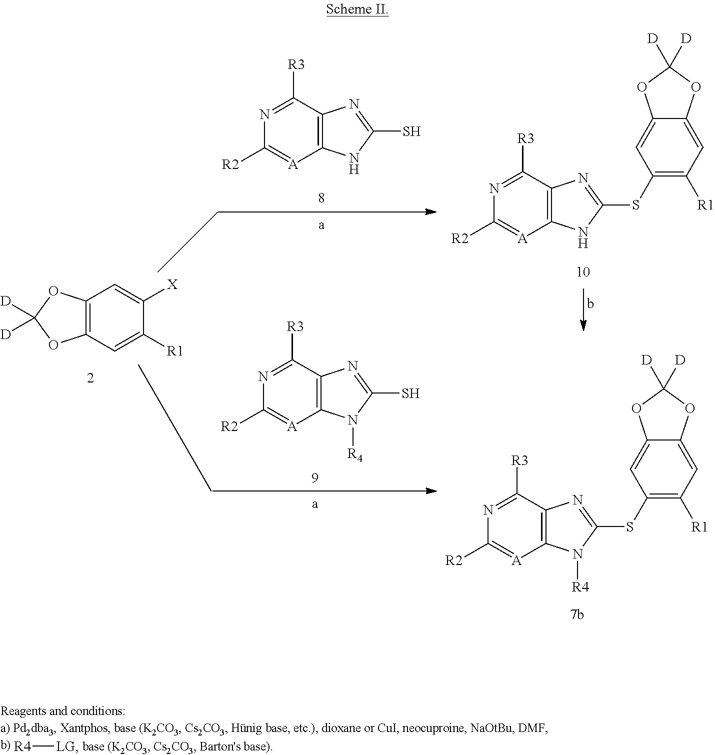

The Scheme II shows two general methods for the synthesis of 7b from intermediate 2. The first route involves a palladium or copper catalyzed C-S bond formation (WO 2009/06503 and J. Org. Chem. 2004, 69, 3230, the contents of which are incorporated herein in their entirety) of 2 with readily available 8 to provide 10 and the subsequent alkylation of 10 with alkylating agents (R4-LG). Alternatively, the second route utilizes alkylated compound 9 directly for the palladium or copper catalyzed C—S bond formation of reaction with 2. These two synthetic routes are somewhat complementary and can be judiciously chosen on the basis of the structure of target compound. These synthetic routes are also applicable to the synthesis of 7a.

### Specific Synthetic Methods

Scheme 3 illustrates a method of making (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-2-hydroxypropan-1-one, one of the compounds of the present invention, according to some embodiments of the invention:

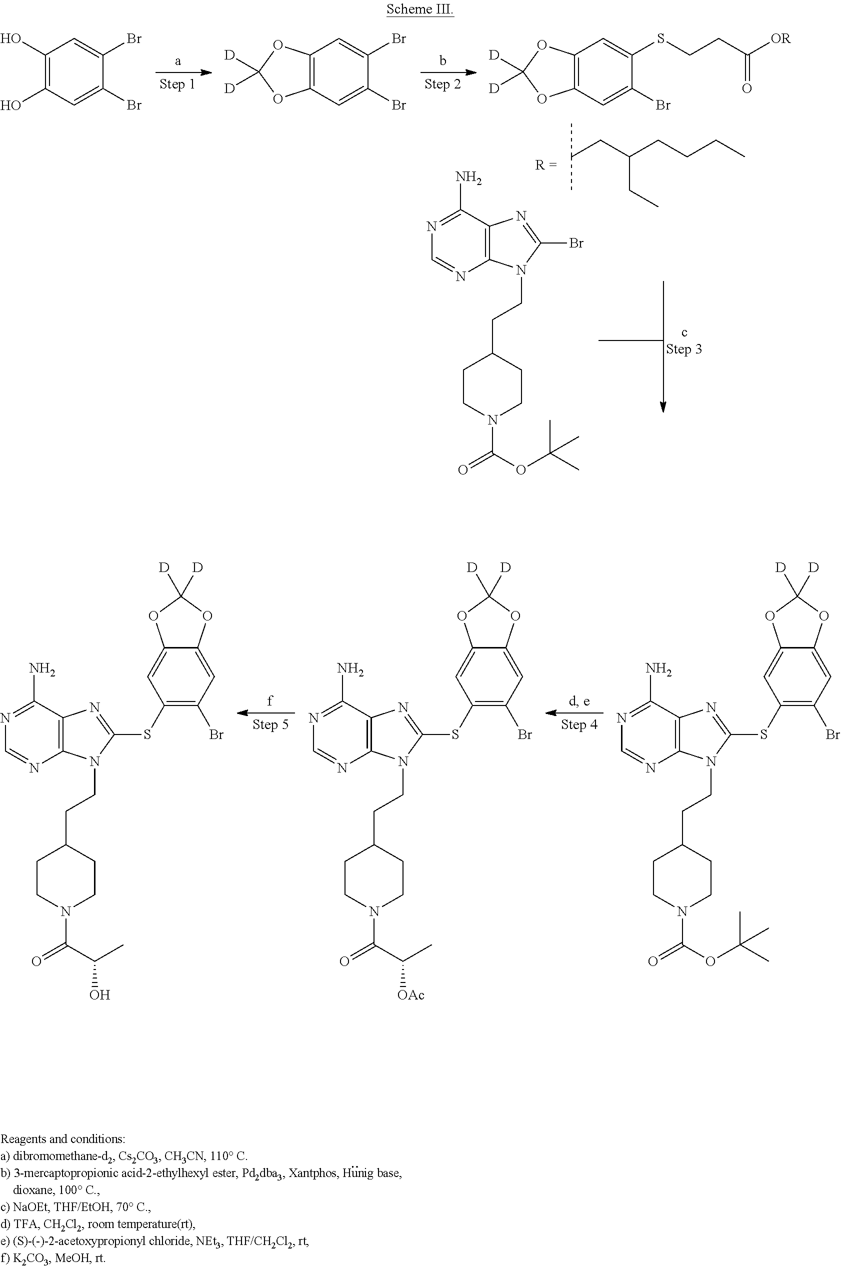

Example I provides a detailed example of a procedure of Scheme III.

### Example Compounds

Step 1: 5,6-Dibromo-2,2-d₂-benzo[d][1,3]dioxole: To a solution of 4,5-dibromobenzene-1,2-diol (667 mg, 2.24 mmol, 90% tech) in CH₃CN (9 mL) was added Cs₂CO₃ (1.1 g, 3.4 mmol). After the mixture had been stirred for 15 min, dibromomethane-d₂ (236 μL, 3.36 mmol, 99.0 atom % D) was added. The resulting mixture was heated at 110° C. for 10 h and upon completion the mixture was diluted with CH₂Cl₂, filtered, and washed with CH₂Cl₂. The combined filtrates were washed with brine, dried (Na₂SO₄), filtered, and concentrated under vacuum. The residue was purified by flash column chromatography (SiO₂, EtOA/hexane, 0 to 10%) to afford the title compound (505 mg, 80%); ¹H NMR (CDCl₃) δ 7.07 (2H); GC/MS: m/z 280 (M⁺).

Step 2: 2-Ethylhexyl 3-[(6-bromo-2,2-d₂-benzo[d][1,3]dioxol-5-yl)sulfanyl]propanoate: 5,6-Dibromo-2,2-d₂-benzo[d][1,3]dioxole (500 mg, 1.77 mmol), 3-mercaptopropionic acid-2-ethylhexylester (407 mg, 1.86 mmol), Pd₂dba₃ (33 mg, 0.02 mmol), Xantphos (41 mg, 0.04 mmol), and Hünig base (618 μL, 3.55 mmol) were placed in a vial, and degassed dioxane (7 mL) was added to the vial. After stirring for 10 h at 100° C., the mixture was cooled to rt, diluted with CH₂Cl₂, filtered, washed with CH₂Cl₂. The combined filtrates were concentrated under vacuum and the residue was purified by flash column chromatography on SiO₂ (EtOAc/hexane, 0 to 10%) to provide the title compound (315 mg, 42%); ¹H NMR (CDCl₃) δ 7.06 (s, 1H), 6.95 (s, 1H), 4.05-3.98 (m, 2H), 3.11 (d, J=8.0 Hz, 2H), 2.61 (d, J=8.0 Hz, 2H), 1.57 (m, 1H), 1.38-1.24 (m, 8H), 0.89 (two t, J=7.6 Hz, 6H); GC/MS: m/z 418 (M⁺).

Step 3: tert-Butyl 4-(2-{6-amino-8-[(6-bromo-2,2-d₂-benzo[d][1,3]dioxol-5-yl)sulfanyl]-9H-purin-9-yl}ethyl)piperidine-1-carboxylate: To a solution of 2-ethylhexyl 3-[(6-bromo-2,2-d₂-benzo[d][1,3]dioxol-5-yl)sulfanyl]propanoate (300 mg, 0.715 mmol) in THF (2 mL) was treated with NaOEt (270 μL, 0.715 mmol, 2.69 M in EtOH) at 0° C. After stirring for 2 h at rt, the mixture was concentrated under vacuum and the resulting sodium thiolate was dissolved in a mixture of THF (2 mL) and EtOH (0.2 mL). To the above solution tert-Butyl 4-[2-(6-amino-8-bromo-9H-purin-9-yl)ethyl]piperidine-1-carboxylate (169 mg, 0.40 mmol), which was prepared by procedure previously reported (WO 2009/065035, the contents of which are incorporated herein by reference in their entirety), was added, and the resulting mixture was heated at 70° C. for 10 h. After cooling to rt, the mixture was concentrated under vacuum and the residue was diluted with EtOAc, washed with brine, dried (Na₂SO₄), filtered, and concentrated under vacuum. Purification of the residue by column chromatography on SiO₂ (MeOH/CH₂Cl₂, 0 to 10%) provided the title compound (200 mg, 87%); TOF LC/MS [M+H]⁺ 579.14.

Step 4: (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-1-oxopropan-2-yl acetate: A solution of tent-Butyl 4-(2-{6-amino-8-[(6-bromo-2,2-d₂-benzo[d][1,3]dioxol-5-yl)sulfanyl]-9H-purin-9-yl}ethyl)piperidine-1-carboxylate (190 mg, 0.328 mmol) in CH₂Cl₂ (3 mL) was treated with TFA (487 μL, 6.56 mmol) at 0° C. After stirring for 8 h at rt, the mixture was concentrated under vacuum and the residual TFA was removed by an azeotrope with toluene to afford the Boc deprotected product as a TFA salt which was directly used for the next step without further purification. A suspension of the crude compound (290 mg) in a mixture of THF (3.3 mL) and CH₂Cl₂ (1.3 mL) was treated with NEt₃ (182 μL, 1.31 mmol) at 0° C., followed by (S)-(−)-2-acetoxypropionyl chloride (46 μL, 0.36 mmol). After stirring for 12 h at rt, the mixture was diluted with CH₂Cl₂, washed with brine, dried (Na₂SO₄), filtered, and concentrated under vacuum. The residue was purified by trituration using EtOAc to yield the title compound (147 mg, 75%, two steps); TOF LC/MS [M+H]⁺ 593.11.

Step 5: (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-2-hydroxypropan-1-one: To a solution of (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-1-oxopropan-2-yl acetate (141 mg, 0.238 mmol) in a mixture of MeOH (4 mL) and CH₂Cl₂ (1 mL) was added K₂CO₃ (66 mg, 0.48 mmol) at 0° C. and the mixture was slowly warmed up to rt. After stirring at rt for 6 h, the mixture was diluted with a mixture of CH₂Cl₂/MeOH (10 mL, v/v =9/1), filtered, washed with CH₂Cl₂. The combined filtrates were concentrated under vacuum and the residue was purified by preparative HPLC to provide the title compound (40 mg) in the form of TFA salt after lyophilization; ¹H NMR (DMSO-d₆) δ 8.32 (s, 1H), 7.41 (s, 1H), 6.93 (s, 1H), 4.39 (m, 1H), 4.31 (m, 1H), 4.22 (t, J=7.2 Hz, 2H), 3.92 (bd, J=12.1 Hz, 1H), 2.87 (m, 1H), 2.47 (m, 1H), 1.77-1.70 (m, 2H), 1.63 (q, J=7.2 Hz, 2H), 1.45 (m, 1H),1.16-1.13 (m, 3H), 1.12-0.62 (m, 2H); TOF LC/MS [M+H]⁺ 551.10.

### Biological and Pharmacological Assays and Examples

### Hsp90 Binding Assay:

The binding of exemplary compounds to purified Hsp90 can be assayed by measuring the displacement of BODIPY-labeled geldanamycin (BODIPY-GM) from purified human Hsp90, using a fluorescence polarization assay adapted from Kim et al. (Journal of Biomolecular Screening 2004, 9(5):375-381) Compound dilutions (in 100% DMSO) are added to black-bottom 96-well plates (Greiner; 2% DMSO final), and equal volumes of BODIPY-GM (10 nM final) and purified human Hsp90 (Stressgen, SPP-770; 30 nM final) in assay buffer (20 mM HEPES-KOH pH 7.3, 50 mM KCl, 5 mM MgCl₂, 20 mM Na₂MoO₄, 0.01% NP-40, 0.1 mg/mL bovine gamma globulin [Invitrogen, P2045], 2 mM DTT) are added sequentially to yield a final volume of 50 microliters. Plates are incubated overnight at room temperature. Parallel and perpendicular fluorescence measurements are read (LJL BioSystems Analyst AD plate reader) at excitation/emission wavelengths of 485/530 nm. Background fluorescence (buffer only) is subtracted, and fluorescence polarization (FP) values, expressed in mP units, are calculated from parallel and perpendicular fluorescence readings as follows:

FP=(parallel−perpendicular)/(parallel+perpendicular)*1000.

Percent inhibition is calculated by normalizing the FP values to those obtained in parallel reactions containing DMSO and subtracting these normalized values from 100%. Intrinsic compound fluorescence is independently monitored, and FP data points confounded by compound fluorescence are excluded from the analysis. In one embodiment, the invention provides compounds of Formula I and II, wherein the compounds have an IC₅₀ as measured by this assay of 10 μM or less, 5μM or less, 1 μM or less, 0.5 μM or less, 0.25 μM or less, or 0.1 μM or less.

For example, using this assay, (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-2-hydroxypropan-1-one exhibited an IC₅₀ of 0.25 μM (250 nM), and (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-1-oxopropan-2-yl acetate exhibited an IC₅₀ of 0.078 μM.

### Her2-Luciferase Assay:

HCT116 cells stably transfected with a Her2 (kinase domain)-Luciferase fusion are seeded into black 96-well plates at 10,000 cells per well in 100 microliters (DMEM supplemented with 10% serum) and incubated overnight. Compound dilutions (in 100% DMSO) are added to individual wells (0.4% DMSO final), and plates are incubated for four hours. Plates are equilibrated to room temperature (5 min), and 100 microliters Steady-Glo reagent (Promega #E2520) is added per well, and plates are incubated at room temperature for 5 minutes. Luminescence is then measured (TopCount, Perkin-Elmer).

### Cytotoxicity Assay:

HCT116 cells are seeded into black 96-well plates at 5,000 cells per well in 100 microliters (DMEM supplemented with 10% serum) and are incubated overnight. Compound dilutions (in 100% DMSO) are added to individual wells (0.4% DMSO final), and plates are incubated for 72 hours. Plates are equilibrated to room temperature (5 min). Fifty microliters lysis buffer followed by 50 microliters substrate solution (ATPLite [2 step], Perkin-Elmer, #601941) is added to each well, and plates are incubated at room temperature 5 minutes. Luminescence is then measured (TopCount, Perkin-Elmer).

For example, using this assay, (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-2-hydroxypropan-1-one exhibited an IC₅₀ of 1.2 μM, and (2S)-1-[4-(2-{6-amino-8-[(6-bromo-2,2-d₂-benzo[d][1,3]dioxol-5-yl)sulfanyl]-9H-purin-9-yl}ethyl)piperidin-1-yl]-1-oxopropan-2-yl acetate exhibited an IC₅₀ of 0.73 μM.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A compound having a structure according to Formula I: 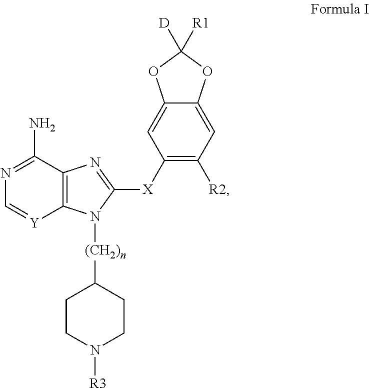
wherein:
D is deuterium;
R1 is hydrogen or deuterium;
R2 is hydrogen, halo, hydroxyl, methoxy, trihalomethoxy, trihalomethyl, C1-C6 alkyl, —CH₂OH, CHF₂, CH₂F, cyano, nitro, amino, aminoalkyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, or N-thiocarbamyl;
R3 is hydrogen or -C(═O)R5, wherein R5 is selected from hydrogen, C₁₋₆ alkyl, aryl, and cycloalkyl, which are optionally substituted with one or more groups selected from halo, hydroxyl, thiol, alkylthio, arylthio, cyano, haloalkyl, alkoxy, amino, C-amido, N-amido, sulfonyl, sulfonamide, and heteroaryl;
X is CH₂, NH, NCH₃, NCH₂CH₃, NCH(CH₃)₂, O, or S;
Y is N or CH; and
n is 0 to 4;
and pharmaceutically acceptable salts thereof.

2. A compound according to claim 1, wherein R1 is deuterium.

3. A compound according to claim 1, wherein R2 is halo or amino.

4. A compound according to claim 3, wherein R2 is chloro or bromo.

5. A compound according to claim 3, wherein R2 is —N(CH₃)₂.

6. A compound according to claim 1, wherein n is 2.

7. A compound according to claim 1, wherein X is S.

8. A compound according to claim 1, wherein Y is N.

9. A compound according to claim 1, wherein R3 is selected from

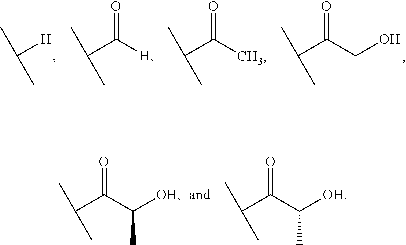

10. A compound according to claim 1, wherein:
R1 is deuterium;
R2 is halo or amino;
R3 is selected from 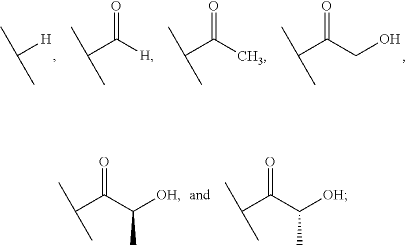
X is S;
Y is N or CH; and
n is 2;
and pharmaceutically acceptable salts thereof.

11. A pharmaceutical composition comprising a compound according to claim 1 and a pharmaceutically acceptable carrier or excipient.

12. A method of treating an Hsp90 inhibitor-sensitive cancer comprising identifying a patient in need of such treatment and administering to said patient a therapeutically effective amount of a compound according to claim 1.

13. The method of claim 12, wherein said Hsp90 inhibitor-sensitive cancer is selected from Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

14. A compound that is (2S)-1-[4-(2-{6-amino-8-[(6-bromo-2,2-d₂-benzo[d][1,3]dioxol-5-yl)sulfanyl]-9H-purin-9-yl}ethyl)piperidin-l-yl]-2-hydroxypropan-1-one, and pharmaceutically acceptable salts thereof.

15. A pharmaceutical composition comprising a compound according to claim 14 and a pharmaceutically acceptable carrier or excipient.

16. A method of treating an Hsp90 inhibitor-sensitive cancer comprising identifying a patient in need of such treatment and administering to said patient a therapeutically effective amount of a compound according to claim 14.

17. A compound having a structure according to Formula II: 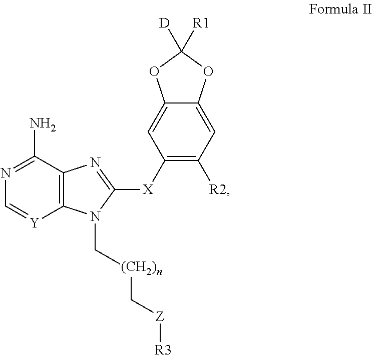
wherein:
D is deuterium;
R1 is hydrogen or deuterium;
R2 is hydrogen, halo, hydroxyl, methoxy, trihalomethoxy, trihalomethyl, C1-C6 alkyl, —CH₂OH, CHF₂, CH₂F, cyano, nitro, amino, aminoalkyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, and N-thiocarbamyl;
R3 is hydrogen, alkyl, alkenyl, alkynyl, amino, cyano, carbocycle, or heterocycle;
X is CH₂, NH, NCH₃, NCH₂CH₃, NCH(CH₃)₂, O, or S;
Y is N or CH;
Z is optionally present, and when present is O, S, CH₂, CHR4, NH, or NR4, wherein R4 is independently selected from H, alkyl, carbocycle, heterocycle, amino, aminoalkyl, carbonyl, C-amido, N-amido, C-amidoalkyl, O-carboxy, C-carboxy, ester, C-carboxy salt, acetyl, carboxyalkyl, carboxyalkyl salt, carboxylic acid, O-carbamyl, N-carbamyl, O-thiocarbamyl, and N-thiocarbamyl; and
wherein n is 0 to 3;
and pharmaceutically acceptable salts thereof.

18. A compound according to claim 17, wherein:
n is 0 or 1;
Y is N or CH;
R2 is halo or amino;
Z is NH or CHR4;
R4, when present, is cyclopropyl, cyclobutyl, cyclopentyl, cyclohexyl, or heterocycle;
R3 is selected from —NH2, isopropyl, tert-butyl, 2,2-dimethyl propyl, 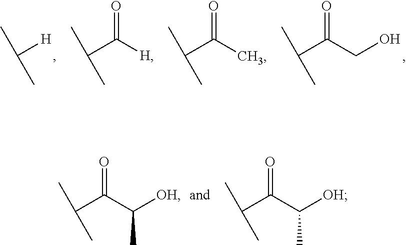
and pharmaceutically acceptable salts thereof.

19. A pharmaceutical composition comprising a compound according to claim 17 and a pharmaceutically acceptable carrier or excipient.

20. A method of treating an Hsp90 inhibitor-sensitive cancer comprising identifying a patient in need of such treatment and administering to said patient a therapeutically effective amount of a compound according to claim 17.
